SEQUENCE LISTING (5 sequences)

SEQ ID NO: 1 - protein, 976 aa, Homo sapiens [sequence omitted]

SEQ ID NO: 2 - DNA, 30 bp, Homo sapiens
  Primer
aagaagagatggtacctcgaggggtgaccc

SEQ ID NO: 3 - DNA, 33 bp, Homo sapiens
  Primer
ctgcttcgcggccgcgttaactcttctcaacca

SEQ ID NO: 4 - DNA, 20 bp, Homo sapiens
  Primer
agctcgtttagtgaaccgtc

SEQ ID NO: 5 - DNA, 20 bp, Homo sapiens
  Primer
gtcagacaaaatgatgcaac

(19) United States
(12) Patent Application Publication
(10) Pub. No.: US 2005/0176687 A1
(43) Pub. Date: Aug. 11, 2005

(54) Use of tyrosine kinase inhibitors for treating autoimmune diseases

(76) Inventors: Alain Moussy, Paris (FR); Jean-Pierre Kinet, Lexington, MA (US)

Correspondence Address: SUGHRUE MION, PLLC, 2100 PENNSYLVANIA AVENUE, N.W., SUITE 800, WASHINGTON, DC, 20037, US

(21) Appl. No.: 10/482,758

(22) PCT Filed: Jun. 28, 2002
(86) PCT No.: PCT/IB02/03302

(30) Foreign Application Priority Data
Jun. 29, 2001 (US) 60301405
Jun. 29, 2001 (US) 60301410
Jun. 29, 2001 (US) 60301409
Dec. 20, 2001 (US) 60341273

(51) Int. Cl. A61K 31/66; A61K 31/519; A61K 31/517

(52) U.S. Cl. 514/114; 514/256; 514/264.1; 514/266.1; 514/249; 514/359; 514/357; 514/406; 514/312; 514/418

(57) ABSTRACT

The present invention relates to a method for treating autoimmune diseases, more particularly selected from the group consisting of multiple sclerosis, ulcerative colitis, Chron's disease, rheumatoid arthritis and polyarthritis, scleroderma, lupus erythematosus, dermatomyositis, pemphigus, polymyositis, vasculitis, as well as graft-versus host diseases, comprising administering a compound capable of depleting mast cells to a mammal in need of such treatment. Such compounds can be chosen from tyrosine kinase inhibitors and more particularly on-toxic, selective and potent c-kit inhibitors. Preferably, said inhibitor is unable to promote death of IL-3 dependent cells cultured in presence of IL-3.

The present invention relates to a method for treating autoimmune diseases, more particularly selected from the group consisting of multiple sclerosis, ulcerative colitis, Crohn's disease, rheumatoid arthritis and polyarthritis, scieroderma, lupus erythematosus, dermatomyositis, pemphigus, polymyositis, vasculitis, as well as graft-versus host diseases, comprising administering a compound capable of depleting mast cells to a mammal in need of such treatment. Such compounds can be chosen from tyrosine kinase inhibitors and more particularly non-toxic, selective and potent c-kit inhibitors. Preferably, said inhibitor is unable to promote death of IL-3 dependent cells cultured in presence of IL-3.

Autoimmune diseases arise when immune system cells (lymphocytes, macrophages) become sensitized against the “self”. Lymphocytes TH and CTL, B lymphocytes as well as macrophages are usually under control in this system. But, a misdirection of the system toward the body's own tissues may happen for still unexplained triggers. In other words, autoimmune disorders occur when the normal control process is disrupted.

The hypothesis is that lymphocytes recognize at some point an antigen which mimics the “self” and a cascade of activation of different components of the immune system takes place, ultimately leading to tissue destruction. Genetic predisposition has also been postulated to be responsible for autoimmune disorders. Autoimmune diseases can affect connective tissue, but it can also affect the nerves, muscles, endocrine system, and the gastrointestinal system.

According to the American Autoimmune Related Diseases Association (AARDA), autoimmune diseases must be regarded “as a united group of disorders”. Indeed, the presence of one autoimmune disease in one patient implies the possibility that a second or third autoimmune disease may occur in the same individual or in other members of the same family.

As suggested by the AARDA, an effective treatment of autoimmune disease requires the identification and turning off these disease-producing T cells. But as of today, such a cure remain elusive.

Typically, these diseases are treated with corticosteroids and immunosuppressant medications (including cyclophosphamide or azathioprine) to reduce the immune response. In addition, U.S. Pat. No. 6,248,368 describes the use of magnesium to treat autoimmune diseases in association with vitamin B6. Compositions containing purified anti-idiotypic antibodies have also been proposed in U.S. Pat. No. 6,231,856 and compositions containing an antisense oligonucleotides targeted to nucleic acids encoding TNFα are mentioned in U.S. Pat. No. 6,228,642 for treating autoimmune diseases.

But, none of the above available treatments are effective and safe for treating autoimmune diseases. In addition, the prolonged use of immunosuppressor drugs lead to adverse side effects and morbidity. Moreover, autoimmunity related disorders require lifetime care and treatment, which is expensive and often lead to the disruption of the lifestyle of patients.

Therefore, the problem is to find alternative solutions to provide a relief and a cure for the numerous patients afflicted with these diseases.

In connection with the present invention, we propose that mast cells are the central players involved in autoimmune diseases. Mast cells (MC) are tissue elements derived from a particular subset of hematopoietic stem cells that express CD34, c-kit and CD13 antigens (Kirshenbaum et al, Blood. 94: 2333-2342, 1999 and Ishizaka et al, Curr Opin Immunol. 5: 93743, 1993). Immature MC progenitors circulate in the bloodstream and differentiate in tissues. These differentiation and proliferation processes are under the influence of cytokines, one of utmost importance being Stem Cell Factor (SCF), also termed Kit ligand (KL), Steel factor (SL) or Mast Cell Growth Factor (MCGF). SCF receptor is encoded by the protooncogene c-kit, that belongs to type III receptor tyrosine kinase subfamily (Boissan and Arock, J Leukoc Biol. 67: 13548, 2000). This receptor is also expressed on others hematopoietic or non hematopoietic cells. Ligation of c-kit receptor by SCF induces its dimerization followed by its transphosphorylation, leading to the recruitement and activation of various intracytoplasmic substrates. These activated substrates induce multiple intracellular signaling pathways responsible for cell proliferation and activation (Boissan and Arock, 2000). Mast cells are characterized by their heterogeneity, not only regarding tissue location and structure but also at the functional and histochemical levels (Aldenborg and Enerback., Histochem. J. 26: 587-96, 1994; Bradding et al. J Immunol. 155: 297-307, 1995; Irani et al, J Immunol. 147: 247-53, 1991; Miller et al, Curr Opin Immunol. 1: 63742, 1989 and Welle et al, J Leukoc Biol. 61: 23345, 1997).

Activation of the detrimental immune response to the self is postulated here to results or to be further stimulated from the degranulation of mast cells. Among to cytokines secreted by mast cells, IFNγ is of particular interest. Indeed, it has been observed that IFNγ is responsible for major histocompatibility complex (MHC) associated autoimmune diseases; Hooks et al, (1979) New England J.Med., Vol. 301: 5-8. For example, higher IFNγ levels were shown to correlate with greater severity of disease in SLE patients.

TNF is another cytokine produced by mast cells. More recently, it has been reported that TNF produced by mast cells was involved in the pathogenesis of autoantibody-mediated vasculitis, Watanabe N. et al Blood Dec. 1, 1999;94(11):3855-63. In Biedermann T et al, J Exp Med Nov. 20, 2000;192(10):1441-52, it is shown that mast cells control neutrophil recruitment during T cell-mediated delayed-type hypersensitivity reactions through TNF and macrophage inflammatory protein 2 (MIP-2).

In addition, mast cells are postulated here to participate in the destruction of tissues by releasing a cocktail of different proteases and mediators categorized into three groups: preformed granule-associated mediators (histamine, proteoglycans, and neutral proteases), lipid-derived mediators (prostaglandins, thromboxanes and leucotrienes), and various cytokines (IL-1, IL-2, IL-3, IL-4, IL-5, IL-6, IL-8, TNF-α, GM-CSF, MIP-1a, MIP-1b, MIP-2 and IFN-γ). Then, liberation by activated mast cells of mediators (TNF-α, histamine, leucotrienes, prostaglandines etc . . . ) as well as proteases is proposed here i) to induce and activate components of the immunity involved in autoimmune diseases and ii) to participate in the tissue destruction process. The activation of T and B lymphocytes against the self stimulate mast cells, which in turn release the above mentioned factors further activating components of the autoimmune reaction.

To break the formation of this cycle leading to tissue destruction, the present invention proposes to deplete mast cells using compounds that are substantially specific to mast cells. In this regard, tyrosine kinase inhibitors and more particularly c-kit specific kinase inhibitors are proposed to inhibit mast cell proliferation, survival and activation.

A new route for treating autoimmune diseases is provided, which consists of destroying mast cells playing a role in the pathogenesis of these disorders. It has been found that tyrosine kinase inhibitors and more particularly c-kit inhibitors are especially suited to reach this goal.

### DESCRIPTION

The present invention relates to a method for treating autoimmune diseases comprising administering a compound capable of depleting mast cells to a mammal in need of such treatment.

Said method for treating autoimmune diseases can comprise administering a tyrosine kinase inhibitor to a mammal in need of such treatment.

Tyrosine kinase inhibitors are selected for example from bis monocyclic, bicyclic or heterocyclic aryl compounds (WO 92/20642), vinylene-azaindole derivatives (WO 94/14808) and 1-cycloproppyl-4-pyridyl-quinolones (U.S. Pat. No. 5,330,992), Styryl compounds (U.S. Pat. No. 5,217,999), styryl-substituted pyridyl compounds (U.S. Pat. No. 5,302,606), seleoindoles and selenides (WO 94/03427), tricyclic polyhydroxylic compounds (WO 92/21660) and benzylphosphonic acid compounds (WO 91/15495), pyrimidine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504, U.S. Pat. No. 5,883,116, U.S. Pat. No. 5,883,113, U.S. Pat. No 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940) and aryl and heteroaryl quinazoline (U.S. Pat. No. 5,721,237, U.S. Pat. No. 5,714,493, U.S. Pat. No. 5,710,158 and WO 95/15758).

Preferably, said tyrosine kinase inhibitors are unable to promote death of IL-3 dependent cells cultured in presence of IL-3.

In another embodiment, the invention is directed to a method for treating autoimmune diseases comprising administering a c-kit inhibitor to a mammal in need of such treatment.

Preferably, said c-kit inhibitor is a non-toxic, selective and potent c-kit inhibitor. Such inhibitors can be selected from the group consisting of indolinones, pyrimidine derivatives, pyrrolopyrimidine derivatives, quinazoline derivatives, quinoxaiine derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

Among preferred compounds, it is of interest to focus on pyrimidine derivatives such as N-phenyl-2-pyrimidine-amine derivatives (U.S. Pat. No. 5,521,184 and WO 99/03854), indolinone derivatives and pyrrol-substituted indolinones (U.S. Pat. No. 5,792,783, EP 934 931, U.S. Pat. No. 5,834,504), U.S. Pat. No. 5,883,116, U.S. Pat. No. 5,883,113, U.S. Pat. No. 5,886,020, WO 96/40116 and WO 00/38519), as well as bis monocyclic, bicyclic aryl and heteroaryl compounds (EP 584 222, U.S. Pat. No. 5,656,643 and WO 92/20642), quinazoline derivatives (EP 602 851, EP 520 722, U.S. Pat. No. 3,772,295 and U.S. Pat. No. 4,343,940), 4-amino-substituted quinazolines (U.S. Pat. No. 3,470,182), 4-thienyl-2-(1H)-quinazolones, 6,7-dialkoxyquinazolines (U.S. Pat. No. 3,800,039), aryl and heteroaryl quinazoline (U.S. Pat. No. 5,721,237, U.S. Pat. No. 5,714,493, U.S. Pat. No. 5,710,158 and WO 95/15758), 4-anilinoquinazoline compounds (U.S. Pat. No. 4,464,375), and 4-thienyl-2-(1H)-quinazolones (U.S. Pat. No. 3,551,427).

So, preferably, the invention relates to a method for treating autoimmune diseases comprising administering a non toxic, potent and selective c-kit inhibitor is a pyrimidine derivatives, more particularly N-phenyl-2-pyrimidine-amine derivatives of formula I: 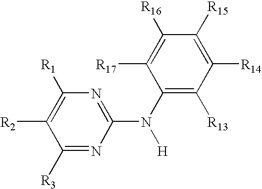
wherein the R1, R2, R3, R13 to R17 groups have the meanings depicted in EP 564 409 B1, incorporated herein in the description.

Preferably, the N-phenyl-2-pyrimidine-amine derivative is selected from the compounds corresponding to formula II: 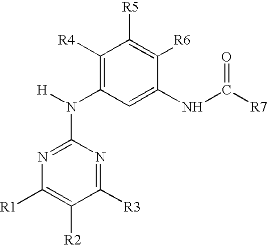
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function.

Preferably, R7 is the following group:

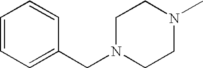

Among these compounds, the preferred are defined as follows:
R1 is a heterocyclic group, especially a pyridyl group,
R2 and R3 are H,
R4 is a C1-C3 alkyl, especially a methyl group,
R5 and R6 are H,
and R7 is a phenyl group bearing at least one substituent, which in turn possesses at least one basic site, such as an amino function, for example the group:

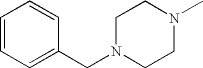

Therefore, in a preferred embodiment, the invention relates to a method for treating autoimmune diseases comprising the administration of an effective amount of the compound known in the art as CGP57148B:
4-(4-méhylpipérazine-1-ylméthyl)-N-[4-méthyl-3-(4-pyridine-3-yl)pyrimidine-2ylamino)phényl]-benzamide corresponding to the following formula:

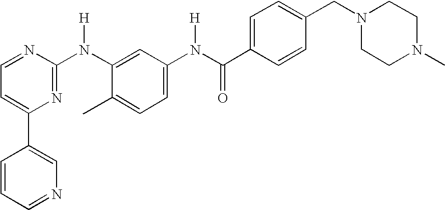

The preparation of this compound is described in example 21 of EP 564 409 and the β-form, which is particularly useful is described in WO 99/03854.

Alternatively, the c-kit inhibitor can be selected from:
indolinone derivatives, more particularly pyrrol-substituted indolinones,
monocyclic, bicyclic aryl and heteroaryl compounds, quinazoline derivatives,
and quinaxolines, such as 2-phényl-quinaxoline derivatives, for example 2-phenyl-6,7-dimethoxy quinaxoline.

In a preferred aspect, the invention contemplated the method mentioned above, wherein said c-kit inhibitor is unable to promote death of IL-3 dependent cells cultured in presence of IL-3.

The following autoimmune diseases as referred herein are contemplated by the present invention:
multiple sclerosis, psoriasis, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, as well as proliferative glomerulonephritis.
graft-versus-host disease or graft rejection in any organ transplantation including kidney, pancreas, liver, heart, lung, and bone marrow.
Other autoimmune diseases embraced by the invention active chronic hepatitis and chronic fatigue syndrome.
subepidermal blistering disorders such as pemphigus.
Vasculitis.

In a preferred embodiment, the method of the invention is applicable to prevent tissue damage and reduce pain in rheumatoid arthritis and in Lupus erythematosis. Lupus is an autoimmune disease in which the immune system becomes overactive and produces antibodies that attack tissues in the body, producing inflammation, redness, pain, and swelling. Lupus is a serious health problem that affects mainly young women.

In another preferred embodiment, the method of the invention is applicable to the treatment of multiple sclerosis. Indeed, a significant increase in the number of mast cells has been observed in the border zones of the plaques. This embodiment is the subject-matter of U.S. 60/601,409 filed by the applicant on Jun. 29, 2001.

In another preferred embodiment, the method of the invention is applicable to the treatment of psoriasis. About 2% of adults have psoriasis, which results in skin growing faster and thicker due to an abnormal immune reaction against some component of the skin.

In another preferred embodiment, the method of the invention is applicable to the treatment of rheumatoid arthritis and polyarthritis, for which the applicant filed U.S. 60/301,410 on Jun. 29, 2001.

In another preferred embodiment, the method of the invention is applicable to the treatment of ulcerative colitis and Crohn's disease, for which the applicant filed U.S. 60/301,405 on Jun. 29, 2001.

In another preferred embodiment, the method of the invention is applicable to the treatment of Dermatomyositis, which an acquired muscle diseases also called inflammatory myopathies. Dermatomyositis is characterized by a rash accompanying, or more often, preceding muscle weakness.

In still another preferred embodiment, the method of the invention is applicable to the treatment of subepidermal blistering disorders. The following subepidermal blistering disorders as referred herein are contemplated by the present invention: aphthous ulcers, and several bullous diseases such as pemphigus, bullous pemphigoid and cicatricial pemphigoid.

The method as depicted above is particularly useful for treating Pemphigus vulgaris. In this disorder, lesions occur in the mouth, as well as on the chest, scalp, periumbilical, and various other areas of the skin. Oral lesions have also been observed. This form of the disease can involve the oropharynx and other mucosal surfaces; this why the invention contemplates compositions for topical as well as oral administration suitable to reach the particular tissues indicated above.

The method as depicted above is particularly useful for treating Pemphigus vegetans, in which vegetating legions and pustules form. Pustules are the result of a super-infection at the edges of the broken bullae. In this regard, antibiotics can be used concomitant with tyrosine kinase inhibitors, for example with c-kit inhibitors. Among antibiotics, the preferred ones are selected from dapsone, azathioprine, erythromycin, propionylerythromycin, neomycin, gentomycin, tobramycin, and mechlocycline. At last, hyperkeratosis, pseudoepitheliomatous hyperplasia, and papillomatosis have also been observed in this disease and will be treated accordingly.

The method as depicted above is also particularly useful for treating Pemphigus foliaceus, which symptoms include crusting, scales, erosion, and excoriations.

The method as depicted above is also particularly useful for treating Pemphigus erythematosus. Here, lesions are lupus-like butterfly rash as well as bullous and seborrheic dermatitis-like lesions.

The method as depicted above is also particularly useful for treating Vasculitis which involves inflammation in blood vessels of various sizes from the aorta to the smallest blood vessels in the skin. This group of diseases encompasses Giant Cell Arteritis, Polymyalgia Rheumatica, Wegener's Granulomatosis, Polyarteritis Nodosa, Hypersensitivity Vasculitis, Rheumatoid Vasculitis, Microscopic Polyangiitis, Buerger's Disease Kawasaki's Disease as well as Vasculitis caused by infection or allergy.

Preferably, the methods as depicted above are applicable for preventing and/or treating autoimmune diseases in human.

In a further embodiment, c-kit inhibitors as mentioned above are inhibitors of activated c-kit. In frame with the invention, the expression “activated c-kit” means a constitutively activated-mutant c-kit including at least one mutation selected from point mutations, deletions, insertions, but also modifications and alterations of the natural c-kit sequence (SEQ ID N^{o}1). Such mutations, deletions, insertions, modifications and alterations can occur in the transphosphorylase domain, in the juxtamembrane domain as well as in any domain directly or indirectly responsible for c-kit activity. The expression “activated c-kit” also means herein SCF-activated c-kit. Preferred and optimal SCF concentrations for activating c-kit are comprised between 5.10⁻⁷ M and 5.10⁻⁶ M, preferably around 2.10⁻⁶ M. In a preferred embodiment, the activated-mutant c-kit in step a) has at least one mutation proximal to Y823, more particularly between amino acids 800 to 850 of SEQ ID No1 involved in c-kit autophosphorylation, notably the D816V, D816Y, D816F and D820G mutants. In another preferred embodiment, the activated-mutant c-kit in step a) has a deletion in the juxtamembrane domain of c-kit. Such a deletion is for example between codon 573 and 579 called c-kit d(573-579). The point mutation V559G proximal to the juxtamembrane domain c-kit is also of interest.

In this regard, the invention contemplates a method for treating autoimmune diseases comprising administering to a mammal in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

This screening method can further comprise the step consisting of testing and selecting a subset of compounds identified in step b) that are inhibitors of mutant activated c-kit (for example in the transphosphorylase domain), which are also capable of inhibiting SCF-activated c-kit wild.

Alternatively, in step a) activated c-kit is SCF-activated c-kit wild.

A best mode for practicing this method consists of testing putative inhibitors at a concentration above 10 μM in step a). Relevant concentrations are for example 10, 15, 20, 25, 30, 35 or 40 μM.

In step c), IL-3 is preferably present in the culture media of IL-3 dependent cells at a concentration comprised between 0.5 and 10 ng/ml, preferably between 1 to 5 ng/ml.

Examples of IL-3 dependent cells include but are not limited to:
cell lines naturally expressing and depending on c-kit for growth and survival. Among such cells, human mast cell lines can be established using the following procedures: normal human mast cells can be infected by retroviral vectors containing sequences coding for a mutant c-kit comprising the c-kit signal peptide and a TAG sequence allowing to differentiate mutant c-kits from c-kit wild expressed in hematopoetic cells by means of antibodies.

This technique is advantageous because it does not induce cellular mortality and the genetic transfer is stable and gives satisfactory yields (around 20%). Pure normal human mast cells can be routinely obtained by culturing precursor cells originating from blood obtained from human umbilical vein. In this regard, heparinated blood from umbilical vein is centrifuged on a Ficoll gradient so as to isolate mononucleated cells from other blood components. CD34+ precursor cells are then purified from the isolated cells mentioned above using the immunomagnetic selection system MACS (Miltenyi biotech). CD34+ cells are then cultured at 37° C. in 5% CO₂ atmosphere at a concentration of 10⁵ cells per ml in the medium MCCM (α-MEM supplemented with L-glutamine, penicillin, streptomycin, 5 10⁻⁵ M β-mercaptoethanol, 20% veal fetal serum, 1% bovine albumin serum and 100 ng/ml recombinant human SCF. The medium is changed every 5 to 7 days. The percentage of mast cells present in the culture is assessed each week, using May-Grünwal Giemsa or Toluidine blue coloration. Anti-tryptase antibodies can also be used to detect mast cells in culture. After 10 weeks of culture, a pure cellular population of mast cells (>98%) is obtained.

It is possible using standard procedures to prepare vectors expressing c-kit for transfecting the cell lines established as mentioned above. The cDNA of human c-kit has been described in Yarden et al., (1987) EMBO J.6 (11), 3341-3351. The coding part of c-kit (3000 bp) can be amplified by PCR and cloned, using the following oligonucleotides:

| | |
|---|---|
| 5′AAGAAGAGATGGTACCTCGAGGGGTGACCC3′ | (SEQ ID No2) |
| sens | |
| 5′CTGCTTCGCGGCCGCGTTAACTCTTCTCAACCA3′ | (SEQ ID No3) |
| antisens | |

The PCR products, digested with Not1 and Xho1, has been inserted using T4 ligase in the pFlag-CMV vector (SIGMA), which vector is digested with Not1 and Xho1 and dephosphorylated using CIP (Biolabs). The pFlag-CMV-c-kit is used to transform bacterial clone XL1-blue. The transformation of clones is verified using the following primers:

| | |
|---|---|
| 5′AGCTCGTTTAGTGAACCGTC3′ | (SEQ ID No4) |
| sens, | |
| 5′GTCAGACAAAATGATGCAAC3′ | (SEQ ID No5) |
| antisens. | |

Directed mutagenesis is performed using relevant cassettes is performed with routine and common procedure known in the art.

The vector Migr-1 (ABC) can be used as a basis for constructing retroviral vectors used for transfecting mature mast cells. This vector is advantageous because it contains the sequence coding for GFP at the 3′ and of an IRES. These features allow to select cells infected by the retrovirus using direct analysis with a fluorocytometer. As mentioned above, the N-terminal sequence of c-kit c-DNA can be modified so as to introduce a Flag sequence that will be useful to discriminating heterogeneous from endogenous c-kit.

Other IL-3 dependent cell lines that can be used include but are not limited to:
BaF3 mouse cells expressing wild-type or mutated form of c-kit (in the juxtamembrane and in the catalytic sites) are described in Kitayama et al, (1996), Blood 88, 995-1004 and Tsujimura et al, (1999), Blood 93, 1319-1329.
IC-2 mouse cells expressing either c-kit^{WT} or c-kit^{D814Y} are presented in Piao et al, (1996), Proc. Natl. Acad. Sci. USA 93, 14665-14669.

IL-3 independent cell lines are:
HMC-1, a factor-independent cell line derived from a patient with mast cell leukemia, expresses a juxtamembrane mutant c-kit polypeptide that has constitutive kinase activity (Furitsu T et al, J Clin Invest. 1993;92:1736-1744; Butterfield et al, Establishment of an immature mast cell line from a patient with mast cell leukemia. Leuk Res. 1988;12:345-355 and Nagata et al, Proc Natl Acad Sci USA. 1995;92:10560-10564).
P815 cell line (mastocytoma naturally expressing c-kit mutation at the 814 position) has been described in Tsujimura et al, (1994), Blood 83, 2619-2626.

The extent to which component (ii) inhibits activated c-kit can be measured in vitro or in vivo. In case it is measured in vivo, cell lines expressing an activated-mutant c-kit, which has at least one mutation proximal to Y823, more particularly between amino acids 800 to 850 of SEQ ID No1 involved in c-kit autophosphorylation, notably the D816V, D816Y, D816F and D820G mutants, are preferred.

Example of cell lines expressing an activated-mutant c-kit are as mentioned.

In another preferred embodiment, the method further comprises the step consisting of testing and selecting compounds capable of inhibiting c-kit wild at concentration below 1 μM. This can be measured in vitro or in vivo.

Therefore, compounds are identified and selected according to the method described above are potent, selective and non-toxic c-kit wild inhibitors.

Alternatively, the screening method as defined above can be practiced in vitro. In this regard, the inhibition of mutant-activated c-kit and/or c-kit wild can be measured using standard biochemical techniques such as immunoprecipitation and western blot. Preferably, the amount of c-kit phosphorylation is measured.

In a still further embodiment, the invention contemplates a method for treating autoimmune diseases as depicted above wherein the screening comprises:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

Here, the extent of cell death can be measured by 3H thymidine incorporation, the trypan blue exclusion method or flow cytometry with propidium iodide. These are common techniques routinely practiced in the art.

The method according to the invention includes preventing, delaying the onset and/or treating autoimmune diseases in mammals, especially in human.

In the method defined above, any compound capable of depleting mast cells can be used. Such compounds can belong to, as explicated above, tyrosine kinase inhibitors, such as c-kit inhibitors, but are not limited to any particular family so long as said compound shows capabilities to deplete mast cells. Depletion of mast cells can be evaluated using for example one of the mast cell lines depicted above using routine procedure.

Best compounds are compounds exhibiting the greatest selectivity.

Control cell lines include other hematopoeitic cells that are not mast cells or related cells or cell lines. These control cell lines include SCF independent expanded human CD34+ normal cells. These control cells also include but are not limited to the human T lymphocyte Jurkat cell line (ATCC N^{o} TIB-152 and mutant cell lines derived thereof), the human B lymphocyte Daudi or Raji cell line (ATCC N^{o} CCL-213 and CCL-86 respectively), the human monocytic U 937 cell line (ATCC N^{o} CRL-1593.2) and the human HL-60 cell line (ATCC N^{o} CCL-240) and mutant cell lines derived thereof CRL-2258 and CRL-2392).

Such compounds can be identified using with a method for identifying compounds capable of depleting mast cells, said compound being non-toxic for cell types other than mast cells, comprising the step consisting of:
a) culturing mast cells in vitro in a culture medium suitable for mast cells,
b) adding to said culture medium at least one compound to be tested and incubating said cells for a prolonged period of time,
c) selecting compounds that promote mast cells death,
d) identifying a subset of compounds selected in step c) that are unable to promote death of cells selected from the above mentioned control cell lines.

Therefore, the invention embraces the use of the compounds defined above to manufacture a medicament for treating autoimmune diseases such as multiple sclerosis, psoriasis, intestine inflammatory disease, ulcerative colitis, Crohn's disease, rheumatoid arthritis and polyarthritis, dermatomyositis, polymyositis, Sjogren's syndrome, nodular panarteritis, autoimmune enteropathy, proliferative glomerulonephritis, active chronic hepatitis, as well as graft-versus host diseases.

The invention is also directed to the use of the compounds defined above to manufacture a medicament for treating a T cell-mediated disease, preferably one selected from the group consisting of myasthenia gravis, scleroderma, graft-versus-host disease, graft rejection, Graves disease, Addison's disease, autoimmune uveoretinitis, autoimmune thyroidiris, systemic lupus erythematosus, discoid lupus erythematosus, cutaneous lupus, local and systemic scieroderma, psoriasis, dermatomyositis, and primary biliary cirrhosis.

More particularly, the invention concerns the use of the compounds defined above to manufacture a medicament for treating and/or preventing tissue damage and to reduce pain in Lupus erythematosis.

Compounds as defined above can also be used to manufacture a medicament to prevent or treat graft-versus-host disease or graft rejection in any organ transplantation including kidney, pancreas, liver, heart, lung and bone marrow.

Compounds as defined above can also be used to manufacture a medicament to prevent or treat polymyositis, Sjogren's syndrome, nodular panarteritis, autoimmune enteropathy, proliferative glomerulonephritis, active chronic hepatitis and chronic fatigue syndrome.

Compounds as defined above to manufacture a medicament for treating subepidermal blistering disorders such as aphthous ulcers, and several bullous diseases such as pemphigus, bullous pemphigoid and cicatricial pemphigoid.

The pharmaceutical compositions utilized in this invention may be administered by any number of routes including, but not limited to, oral, intravenous, intramuscular, intra-arterial, intramedullary, intrathecal, intraventricular, transdermal, subcutaneous, intraperitoneal, intranasal, enteral, topical, sublingual, or rectal means.

In addition to the active ingredients, these pharmaceutical compositions may contain suitable pharmaceutically-acceptable carriers comprising excipients and auxiliaries which facilitate processing of the active compounds into preparations which can be used pharmaceutically. Further details on techniques for formulation and administration may be found in the latest edition of Remington's Pharmaceutical Sciences (Maack Publishing Co., Easton, Pa.).

Pharmaceutical compositions for oral administration can be formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions, and the like, for ingestion by the patient.

More particularly, the invention relates to a pharmaceutical composition intended for oral or topical administration.

Regarding topical administration, the compositions according to the invention may be presented in the form of a gel, paste, ointment, cream, lotion, liquid suspension aqueous, aqueous-alcoholic or, oily solutions, or dispersions of the lotion or serum type, or anhydrous or lipophilic gels, or emulsions of liquid or semi-solid consistency of the milk type, obtained by dispersing a fatty phase in an aqueous phase or vice versa, or of suspensions or emulsions of soft, semi-solid consistency of the cream or gel type, or alternatively of microemulsions, of microcapsules, of microparticles or of vesicular dispersions to the ionic and/or nonionic type. These compositions are prepared according to standard methods.

The composition according to the invention comprises any ingredient commonly used in dermatology and cosmetic. It may comprise at least one ingredient selected from hydrophilic or lipophilic gelling agents, hydrophilic or lipophilic active agents, preservatives, emollients, viscosity enhancing polymers, humectants, surfactants, preservatives, antioxidants, solvents, and fillers, antioxidants, solvents, perfumes, fillers, screening agents, bactericides, odor absorbers and coloring matter.

As oils which can be used in the invention, mineral oils (liquid paraffin), vegetable oils (liquid fraction of shea butter, sunflower oil), animal oils, synthetic oils, silicone oils (cyclomethicone) and fluorinated oils may be mentioned. Fatty alcohols, fatty acids (stearic acid) and waxes (paraffin, carnauba, beeswax) may also be used as fatty substances.

As emulsifiers which can be used in the invention, glycerol stearate, polysorbate 60 and the PEG-6/PEG-32/glycol stearate mixture are contemplated.

As hydrophilic gelling agents, carboxyvinyl polymers (carbomer), acrylic copolymers such as acrylate/alkylacrylate copolymers, polyacrylamides, polysaccharides such as hydroxypropylcellulose, clays and natural gums may be mentioned, and as lipophilic gelling agents, modified clays such as bentones, metal salts of fatty acids such as aluminum stearates and hydrophobic silica, or alternatively ethylcellulose and polyethylene may be mentioned.

As hydrophilic active agents, proteins or protein hydrolysates, amino acids, polyols, urea, allantoin, sugars and sugar derivatives, vitamins, starch and plant extracts, in particular those of Aloe vera may be used.

As lipophilic active, agents, retinol (vitamin A) and its derivatives, tocopherol (vitamin E) and its derivatives, essential fatty acids, ceramides and essential oils may be used. These agents add extra moisturizing or skin softening features when utilized.

In addition, a surfactant can be included in the composition so as to provide deeper penetration of the compound capable of depleting mast cells, such as a tyrosine kinase inhibitor, preferably a c-kit inhibitor.

Among the contemplated ingredients, the invention embraces penetration enhancing agents selected for example from the group consisting of mineral oil, water, ethanol, triacetin, glycerin and propylene glycol; cohesion agents selected for example from the group consisting of polyisobutylene, polyvinyl acetate and polyvinyl alcohol, and thickening agents.

Chemical methods of enhancing topical absorption of drugs are well known in the art. For example, compounds with penetration enhancing properties include sodium lauryl sulfate (Dugard, P. H. and Sheuplein, R. J., “Effects of Ionic Surfactants on the Permeability of Human Epidermis: An Electrometric Study,” J. Ivest. Dermatol., V.60, pp. 263-69, 1973), lauryl amine oxide (Johnson et. al., U.S. Pat. No. 4,411,893), azone (Rajadhyaksha, U.S. Pat. Nos. 4,405,616 and 3,989,816) and decylmethyl sulfoxide (Sekura, D. L. and Scala, J., “The Percutaneous Absorption of Alkylmethyl Sulfides,” Pharmacology of the Skin, Advances In Biolocy of Skin, (Appleton-Century Craft) V. 12, pp. 257-69, 1972). It has been observed that increasing the polarity of the head group in amphoteric molecules increases their penetration-enhancing properties but at the expense of increasing their skin irritating properties (Cooper, E. R. and Berner, B., “Interaction of Surfactants with Epidermal Tissues: Physiochemical Aspects,” Surfactant Science Series, V. 16, Reiger, M. M. ed. (Marcel Dekker, Inc.) pp. 195-210, 1987).

A second class of chemical enhancers are generally referred to as co-solvents. These materials are absorbed topically relatively easily, and, by a variety of mechanisms, achieve permeation enhancement for some drugs. Ethanol (Gale et. al., U.S. Pat. No. 4,615,699 and Campbell et. al., U.S. Pat. Nos. 4,460,372 and 4,379,454), dimethyl sulfoxide (U.S. Pat. Nos. 3,740,420 and 3,743,727, and U.S. Pat. No. 4,575,515), and glycerine derivatives (U.S. Pat. No. 4,322,433) are a few examples of compounds which have shown an ability to enhance the absorption of various compounds.

Topical composition referred herein are particularly relevant for treating diseases affecting the skin and mucosal membranes. Examples of these disorders include psoriasis, systemic lupus erythematosus, discoid lupus erythematosus, cutaneous lupus, local and systemic scleroderma, and dermatomyositis. This composition comprises a compound capable of depleting mast cells, preferably a tyrosine kinase inhibitor, more particularly a c-kit inhibitor as mentioned above.

Topical composition referred herein are also particularly relevant for treating aphthous ulcers, and several bullous diseases such as pemphigus, bullous pemphigoid and cicatricial pemphigoid since they are affecting especially the skin and mucosal membranes.

As mentioned above, the invention also contemplates a composition suitable for oral administration comprising a compound capable of depleting mast cells, preferably a tyrosine kinase inhibitor, more particularly a c-kit inhibitor for the manufacture of a medicament for the treatment of multiple sclerosis, intestine inflammatory disease, ulcerative colitis, Crohn's disease, rheumatoid arthritis and polyarthritis, myasthenia gravis, polymyositis, graft-versus-host disease, graft rejection, Graves disease, Addison's disease, autoimmune uveoretinitis, autoimmune thyroidiris, primary biliary cirrhosis, Sjogren's syndrome, nodular panarteritis, autoimmune enteropathy, proliferative glomerulonephritis, active chronic hepatitis and chronic fatigue syndrome.

Pharmaceutical compositions suitable for use in the invention include compositions wherein compounds for depleting mast cells, such as tyrosine kinase inhibitors and c-kit inhibitors, are contained in an effective amount to achieve the intended purpose. The determination of an effective dose is well within the capability of those skilled in the art. A therapeutically effective dose refers to that amount of active ingredient, which ameliorates the symptoms or condition. Therapeutic efficacy and toxicity may be determined by standard pharmaceutical procedures in cell cultures or experimental animals, e.g., ED50 (the dose therapeutically effective in 50% of the population) and LD50 (the dose lethal to 50% of the population). The dose ratio of toxic to therapeutic effects is the therapeutic index, and it can be expressed as the ratio, LD50/ED50. Pharmaceutical compositions which exhibit large therapeutic indices are preferred. As mentioned above, a tyrosine kinase inhibitor and more particularly a c-kit inhibitor according to the invention is unable to promote death of IL-3 dependent cells cultured in presence of IL-3.

The invention also contemplates a product comprising at least one compound capable of depleting mast cells, such as a tyrosine kinase inhibitors, more particularly a non-toxic, selective and potent c-kit inhibitor and at least one antibiotic, the preferred ones being selected from cyclophosphamide, methotrexate, dapsone, azathioprine, erythromycin, propionylerythromycin, neomycin, gentomycin, tobramycin, and mechlocycline for simultaneous, separate or sequential use for the treatment of subepidermal blistering disorders, such as pemphigus.

Utility of the invention will further ensue from the detailed description below.

### EXAMPLE 1

### Treatment of Subepidermal Blistering Disorders

Pemphigus affects people across racial and cultural lines. It produces burn-like lesions that will not heal, which results of the loss of intercellular adhesion between the keratinocytes leading to bulla (blister) formation (Sharpe, R. J. in Manual of Clinical Problems in Dermatology, Olbricht, Bigby and Arndt eds., Little Brown & Co., Boston, 1992, pp. 56-60). Pemphigus vulgaris and Pemphigus vegetans are characterized by the formation of blisters above the basal layer of the skin. In Pemphigus foliaceus and Pemphigus erythematosus, blisters are observed just below the stratum corneum. For a review, see Ruocco E, et al, Precautions and suggestions for pemphigus patients, Dermatology 2001;203(3):201-7 and Hertl M, Veldman C, Pemphigus—paradigm of autoantibody-mediated autoimmunity, Int J Fertil Womens Med July-August 2001; 46(4): 190-205.

Current treatments of pemphigus includes corticosteroids and immunosuppressive agents such as cyclophosphamide, azathioprine, methotrexate and cyclosporine-A (Lever, J. Am. Acad. Dermatol. 1979, Vol. 1, pp. 2-31). But, the severity of symptoms and the high mortality associated with pemphigus often lead to hospitalization. In addition, clinically significant bone loss occurs in the vast majority of patients exposed to corticosteroids with a very high risk for vertebral fracture, see Adachi J D, Corticosteroid-induced osteoporosis, Acta Derm Venereol September 1999;79(5):351-5.

Bullous pemphigoid is more prevalent in elderly patients and include large tense blisters, on erythematous or non-erythematous skin or on urticarial plaques. A mortality rate of 10 to 20 percent is reported for the disease, largely due to side-effects from the use of systemic steroid therapy.

Cicatricial Pemphigoid involves primarily the mucous membranes (Baden, L. A., Manual of Clinical Problems in Dermatology, Little, Brown & Co., Boston, 1992, pp. 54). In many cases, this disorder involves desquamative gingivitis and ultimately leads to blindness. Current treatments are as mentioned above and are not satisfactory (Bleicher, supra; Arndt, K. in Fitzpatrick, Eisen, Wolff, Freedberg and Austen, Dermatology in General Medicine, 1987, Vol. 1, McGraw-Hill, Inc., New York, pp. 582-584). Antibiotics can also be used in combinaison with high dose corticosteroids.

A common feature of pemphigus, bullous pemphigoid, cicatricial pemphigoid is the role of proteases in their pathogenesis (Grando, Glukhenky, Drannik, Kostromin and Chemyavsky, Int. J. Tissue React. 1989, Vol. 11, pp. 195-201). This diseases are classified as being mediated by proteases which affect especially the skin and mucosal membranes. In this regard, some proteinase inhibitors have been proposed in the treatment of pemphigus. Furthermore, in U.S. Pat. No. 5,637,616, systemic administration of N-acetylcysteine is proposed for treating these diseases and in U.S. Pat. No. 5,514,714 the use hypericin or pseudohypericin is described for treating pemphigus.

Still, as of today, none of the above available treatments are effective and safe for treating subepidermal blistering disorders. In addition, the prolonged use of immunosuppressor drugs lead to adverse side effects and morbidity.

A long time ago, participation of mast cells was suggested by a sequence of pathologic alterations in which there was progressive mast-cell degranulation and late eosinophil infiltration, Wintroub BU et al, Morphologic and functional evidence for release of mast-cell products in bullous pemphigoid, N EngI J Med Feb. 23, 1978;298(8):417-21. More recently, a significant alterations in mast cell chymase and protease in different bullous diseases has been observed, suggesting mast cell involvement. But, it was thought that this reflected a general inflammation rather than a specific reaction, Kaminska R et al, Mast cells in developing subepidermal bullous diseases: emphasis on tryptase, chymase and protease inhibitors, Acta Derm Venereol September 1999;79(5):35 1-5.

To stop such tissue degradation of skin and mucosal membranes, the present invention proposes to deplete mast cells using compounds that are substantially specific to mast cells. In this regard, tyrosine kinase inhibitors and more particularly c-kit specific kinase inhibitors are proposed to inhibit mast cell proliferation, survival and activation. Evidence of focal and complete degranulation of mast cells was observed in blisters or bullae of patients affected with pemphigus. Besides, it is was observed that B lymphocyte clones produce antibodies directed to the basal membrane of the epidermis. Here, we propose that activation of such detrimental immune response to the self can result from degranulation of mast cells. In addition, this activation of components of immunity goes with the release of proteases that further contribute to the degradation of tissues.

1. A method for treating autoimmune diseases comprising administering a compound capable of depleting mast cells to a mammal in need of such treatment.

2. A method according to claim 1 for treating autoimmune diseases comprising administering a tyrosine kinase inhibitor to a mammal in need of such treatment.

3. A method according to claim 2, wherein said tyrosine kinase inhibitor is unable to promote death of IL-3 dependent cells cultured in presence of IL-3.

4. A method according to claim 2 for treating autoimmune diseases comprising administering a c-kit inhibitor to a mammal in need of such treatment.

5. A method according to claim 4, wherein said c-kit inhibitor is a non-toxic, selective and potent c-kit inhibitor.

6. A method according to claim 5, wherein said inhibitor is selected from the group consisting of indolinones, pyrimidine derivatives, pyrrolopyrimidine derivatives, quinazoline derivatives, quinoxaline derivatives, pyrazoles derivatives, bis monocyclic, bicyclic or heterocyclic aryl compounds, vinylene-azaindole derivatives and pyridyl-quinolones derivatives, styryl compounds, styryl-substituted pyridyl compounds, seleoindoles, selenides, tricyclic polyhydroxylic compounds and benzylphosphonic acid compounds.

7. A method according to claim 5, wherein said inhibitor is selected from the group consisting of:
pyrimidine derivatives, more particularly N-phenyl-2-pyrimidine-amine derivatives.
indolinone derivatives, more particularly pyrrol-substituted indolinones,
monocyclic, bicyclic aryl and heteroaryl compounds,
and quinazoline derivatives.

8. A method according to claim 5, wherein said inhibitor is selected from the group consisting of N-phenyl-2-pyrimidine-amine derivatives having the formula II: 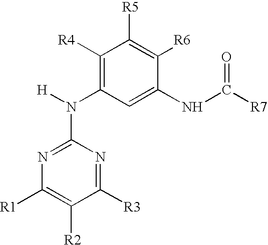
Wherein R1, R2 and R3 are independently chosen from H, F, Cl, Br, I, a C1-C5 alkyl or a cyclic or heterocyclic group, especially a pyridyl group;
R4, R5 and R6 are independently chosen from H, F, Cl, Br, I, a C1-C5 alkyl, especially a methyl group;
and R7 is a phenyl group bearing at least one substituent, which in turn possesses at least one basic site, such as an amino function, preferably the following group:

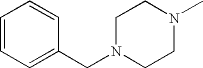

9. A method according to claim 8, wherein said inhibitor is the 4-(4-méhylpipérazine-1-ylméthyl)N-[4-méthyl-3-(4-pyridine-3-yl)pyrimidine-2 ylamino)phényl)-benzamide.

10. A method according to one of claims 4 to 9, wherein said c-kit inhibitor is unable to promote death of IL-3 dependent cells cultured in presence of IL-3.

11. A method according to one of claims 4 to 10, wherein said c-kit inhibitor is an inhibitor of activated c-kit.

12. A method according to claim 11, wherein said inhibitor is capable of inhibiting constitutively activated-mutant c-kit.

13. A method according to claim 11, wherein said activated c-kit inhibitor is capable of inhibiting SCF-activated c-kit.

14. A method for treating autoimmune diseases comprising administering to a mammal in need of such treatment a compound that is a selective, potent and non toxic inhibitor of activated c-kit obtainable by a screening method which comprises:
a) bringing into contact (i) activated c-kit and (ii) at least one compound to be tested; under conditions allowing the components (i) and (ii) to form a complex,
b) selecting compounds that inhibit activated c-kit,
c) testing and selecting a subset of compounds identified in step b), which are unable to promote death of IL-3 dependent cells cultured in presence of IL-3.

15. A method according to claim 14, wherein the screening method further comprises the step consisting of testing and selecting a subset of compounds identified in step b) that are inhibitors of mutant activated c-kit, which are also capable of inhibiting SCF-activated c-kit wild.

16. A method according to claim 14, wherein activated c-kit is SCF-activated c-kit wild in step a).

17. A method according to one of claims 14 to 17, wherein putative inhibitors are tested at a concentration above 10 μM in step a).

18. A method according to one of claims 14 to 18, wherein IL-3 is preferably present in the culture media of IL-3 dependent cells at a concentration comprised between 0.5 and 10 ng/ml, preferably between 1 to 5 ng/ml.

19. A method according to claim 18, wherein IL-3 dependent cells are selected from the group consisting of mast cells, transfected mast cells, BaF3 and IC-2.

20. A method according to one of claims 14 to 19, wherein the extent to which component (ii) inhibits activated c-kit is measured in vitro or in vivo.

21. A method according to one of claims 14 to 20, further comprising the step consisting of testing and selecting compounds capable of inhibiting c-kit wild at concentration below 1 μM.

22. A method according to claim 14 or 21, wherein the testing is performed in vitro or in vivo.

23. A method according to one of claims 14 to 22, wherein the inhibition of mutant-activated c-kit and/or c-kit wild is measured using standard biochemical techniques such as immunoprecipitation and western blot.

24. A method according to one of claims 14 to 23, wherein the amount of c-kit phosphorylation is measured.

25. A method according to one of claims 14 to 24, wherein identified and selected compounds are potent, selective and non-toxic c-kit wild inhibitors.

26. A method for treating autoimmune diseases comprising administering to a mammal in need of such treatment a c-kit inhibitor obtainable by a screening method comprising:
a) performing a proliferation assay with cells expressing a mutant c-kit (for example in the transphosphorylase domain), which mutant is a permanent activated c-kit, with a plurality of test compounds to identify a subset of candidate compounds targeting activated c-kit, each having an IC50<10 μM, by measuring the extent of cell death,
b) performing a proliferation assay with cells expressing c-kit wild said subset of candidate compounds identified in step (a), said cells being IL-3 dependent cells cultured in presence of IL-3, to identify a subset of candidate compounds targeting specifically c-kit,
c) performing a proliferation assay with cells expressing c-kit, with the subset of compounds identified in step b) and selecting a subset of candidate compounds targeting c-kit wild, each having an IC50<10 μM, preferably an IC50<1 μM, by measuring the extent of cell death.

27. A method according to claim 26, wherein the extent of cell death is measured by 3H thymidine incorporation, the trypan blue exclusion method or flow cytometry with propidium iodide.

28. A method according to one of claims 1 to 27 for preventing and/or treating autoimmune diseases in human.

29. A method according to one of claims 1 to 27 for treating multiple sclerosis, psoriasis, subepidermal blistering disorders, intestine inflammatory disease, ulcerative colitis, Crohn's disease, rheumatoid arthritis and polyarthritis, local and systemic scleroderma, systemic lupus erythematosus, discoid lupus erythematosus, cutaneous lupus, dermatomyositis, polymyositis, Sjogren's syndrome, nodular panarteritis, autoimmune enteropathy, proliferative glomerulonephritis, active chronic hepatitis, chronic fatigue syndrome and Vasculitis.

30. A method according to one of claims 1 to 27 for treating graft-versus-host disease or graft rejection in any organ transplantation including kidney, pancreas, liver, heart, lung, and bone marrow.

31. A method according to one of claims 1 to 27 for treating active chronic hepatitis and chronic fatigue syndrome.

32. A method according to one of claims 1 to 27 for treating Lupus erythematosis.

33. A method according to one of claims 1 to 27 for treating psoriasis and subepidermal blistering disorders including aphthous ulcers, and several bullous diseases such as Pemphigus vulgaris, Pemphigus vegetans, Pemphigus foliaceus, and Pemphigus erythematosus, bullous pemphigoid and cicatricial pemphigoid.

34. A method according to one of claims 1 to 27 for treating rheumatoid arthritis and polyarthritis.

35. A method according to one of claims 1 to 27 for treating Dermatomyositis.

36. A method according to one of claims 1 to 27 for treating ulcerative colitis and Crohn's disease.

37. A method according to one of claims 1 to 27 for treating multiple sclerosis.

38. Use of a c-kit inhibitor to manufacture a medicament for treating autoimmune diseases.

39. A composition suitable for topical administration comprising a compound capable of depleting mast cells, preferably a tyrosine kinase inhibitor, more particularly a c-kit inhibitor for the treatment of psoriasis, systemic lupus erythematosus, discoid lupus erythematosus, cutaneous lupus, local and systemic scleroderna, dermatomyositis and Vasculitis.

40. A composition suitable for oral administration comprising a compound capable of depleting mast cells, preferably a tyrosine kinase inhibitor, more particularly a c-kit inhibitor for the treatment of multiple sclerosis, intestine inflammatory disease, ulcerative colitis, Crohn's disease, rheumatoid arthritis and polyarthritis, myasthenia gravis, polymyositis, graft-versus-host disease, graft rejection, Graves disease, Addison's disease, autoimmune uveoretinitis, autoimmune thyroidiris, primary biliary cirrhosis, Sjogren's syndrome, nodular panarteritis, autoimmune enteropathy, proliferative glomerulonephritis, active chronic hepatitis, chronic fatigue syndrome and Vasculitis.

41. A composition suitable for intravenous, intramuscular, intra-arterial, intramedullary, intrathecal, intraventricular, transdermal, subcutaneous, intraperitoneal, enteral, sublingual, or rectal administration comprising a compound capable of depleting mast cells, preferably a tyrosine kinase inhibitor, more particularly a c-kit inhibitor for the treating of autoimmune diseases.

42. A product comprising at least one compound capable of depleting mast cells, such as a tyrosine kinase inhibitors, more particularly a non-toxic, selective and potent c-kit inhibitor and at least one antibiotic, preferably selected from dapsone, azathioprine, erythromycin, propionylerythromycin, neomycin, gentomycin, tobramycin, and mechlocycline for simultaneous, separate or sequential use for the treatment of subepidermal blistering disorders, such as pemphigus.
